(19) United States
(12) Patent Application Publication
(10) Pub. No.: US 2017/0354403 A1
(43) Pub. Date: Dec. 14, 2017

(54) SURGICAL INSTRUMENT WITH PRESURE SENSING AND VISION FEEDBACK FUNCTIONS

(71) Applicant: Chang Bing Show Chwan Memorial Hospital, Lukang Township (TW)

(72) Inventors: Kai-Che LIU, Lukang Township (TW); Shih-Chi CHAN, Lukang Township (TW); Tong-Wen WANG, Lukang Township (TW); Ivan KU, Lukang Township (TW); Chyi-Hsiang WU, Lukang Township (TW)

(73) Assignee: Chang Bing Show Chwan Memorial Hospital, Lukang Township (TW)

(21) Appl. No.: 15/181,296

(22) Filed: Jun. 13, 2016

(51) Int. Cl. A61B 17/00

(52) U.S. Cl. CPC A61B 17/00234; A61B 2017/00128

(57) ABSTRACT

A surgical instrument with pressure sensing and vision feedback functions has an instrument body and a piezochromic device. The instrument body includes a work piece with a visible surface. The piezochromic device is disposed on the visible surface of the work piece of the instrument body and performs color change when the piezochromic device senses pressure. Therefore, when a doctor performs surgery using the surgical instrument, the doctor can directly see the piezochromic device. When the piezochromic device contacts other surgical instruments or presses organs or tissues within a patient's body, the piezochromic device will change color when the piezochromic device senses the pressure enabling the doctor to adjust the surgical instrument accordingly.

### BACKGROUND OF THE INVENTION

### 1. Field of the Invention

The present invention relates to a surgical instrument and more particularly to a surgical instrument with pressure sensing and vision feedback functions.

### 2. Description of Related Art

When a doctor performs a surgery, a skin of a patient's body is cut to expose internal organs or tissues. The doctor manually operates a surgical instrument, such as a scalpel, scissors, a retractor, a clamp, and so on, to perform the surgery. As technology advances, minimally invasive surgical equipment with a display device is provided for the doctor to perform the surgery. The doctor operates the minimally invasive surgical equipment, such as an endoscope, a Da Vinci robot arm, etc., to perform the surgery and the doctor can see the patient's internal body from surgical images on the display device. Therefore, a generation of a huge wound on the patient's body can be avoided to reduce a chance of wound infection and shorten a recovery time. Conversely, a surgeon for performing the minimally invasive surgery requires a professional training to become familiar with the functions of the surgical equipments. Therefore, the doctor can familiarly perform the minimally invasive surgery.

When the doctor manually performs the surgery in person, multiple different instruments are put on the patient and the doctor has to hold the surgical instruments to move around the wound site. A well-trained doctor can determine whether the surgical instruments hit or press the unwounded organs or the unwounded tissues and adjust the movement properly. However, an inexperienced doctor or a medical intern with less experience may not be able to determine whether the surgical instruments hit or press the unwounded organs or the unwounded tissues, and this may affect the performance of the surgery. For example, an accidental cut or hurt may happen in normal organs, tissues, blood vessels, veins, arteries or nerves. On the other hand, for the minimally invasive surgical equipment, the doctor performs the surgery in the human body indirectly by the minimally invasive surgical instruments rather than manually performs the surgery by the surgical instruments in the human body. Therefore, the doctor does not have a manual tactile feedback and it is hard for the doctor to feel whether the surgical instruments hit or press the unwounded organs or the unwounded tissues.

### SUMMARY OF THE INVENTION

Accordingly, a main objective of the present invention is to provide a surgical instrument with pressure sensing and vision feedback functions to assist a doctor to determine whether the surgical instrument hits other surgical instruments or presses organs or tissues within a patient's body.

The surgical instrument with pressure sensing and vision feedback functions comprises:

an instrument body including a work piece with a visible surface; and

a piezochromic device disposed on the visible surface of the work piece of the instrument body and performing color change when the piezochromic device senses pressure.

According to the structure in the present invention, the work piece of the instrument body can enter the patient's body and the visible surface is a surface where the doctor can directly see during the surgery. Therefore, the doctor can directly see the piezochromic device during the surgery. Moreover, the piezochromic device changes the color when the piezochromic device senses the pressure. When the piezochromic device hits other instruments or presses the organ or the tissue within the patient's body, the piezochromic device changes the color immediately when the piezochromic device senses the pressure for the doctor to directly determine.

Accordingly, regardless that the surgeon is a well-trained doctor or a doctor with less experience or the surgeon is manually operated by the doctor in person or the doctor operates the minimally invasive surgery equipment, the piezochromic device changes the color when the piezochromic device is hit or pressed. The doctor can directly see the piezochromic device to determine whether the color of the piezochromic device is changed or not so as to adjust the surgical movement. The surgical instrument can be efficiently prevented from hitting or pressing unwounded organs or tissues within the patient's body to guarantee that the surgery is successfully performed. Moreover, the structure in the present invention is simple and easy to operate, and the practicability is enhanced.

### BRIEF DESCRIPTION OF THE DRAWINGS

FIG. 1 is a plan view of a surgical instrument in an embodiment of the present invention;

FIG. 2 is a plan view of a piezochromic device in the embodiment of the present invention; and

FIG. 3 is a plan view of the piezochromic device in another embodiment of the present invention.

### DETAILED DESCRIPTION OF THE PREFERRED EMBODIMENT

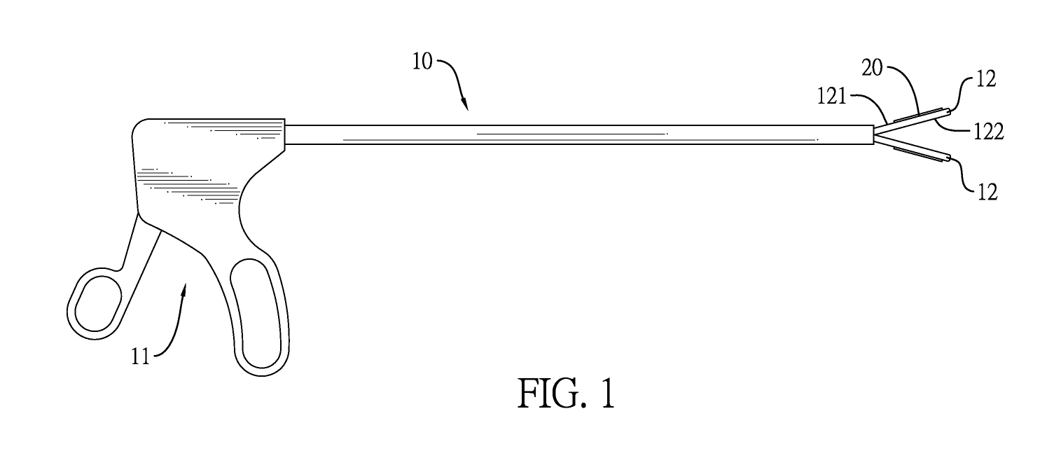

With reference to FIG. 1, a surgical instrument with pressure sensing and vision feedback functions in the present invention includes an instrument body 10 and a piezochromic device 20. It should be noted that the surgical instrument of the present invention is not limited to be a handheld surgical instrument and can also be a surgical instrument for minimally invasive surgery equipment.

The instrument body 10 includes a handle 11 and a work piece 12. The handle 11 can be a handheld component such that medical staffs can hold the handle 11. In another embodiment, the handle 11 can be a connecting portion such that a robotic arm of the minimally invasive surgical equipment can be connected to the handle 11. The work piece 12 is a portion that can enter a patient's body when the instrument body 10 is used during a surgery. The work piece 12 includes a visible surface 121 and the visible surface 121 is a surface where the doctor can directly see during the surgery. For example, the instrument body 10 can be a scalpel, surgical scissors, blood vessel pliers, forceps, a needle holder, tissue forceps, a towel clamp, a right-angle clamp, an intestinal clamp, a stomach clamp, a towing hook, a suction, etc, but it is not limited herein. The instrument body 10 shown in FIG. 1 is a handheld clamp as an example, and the work piece 12 includes a clamp surface 122 for gripping objects and the visible surface 121 is another surface opposite to the clamp surface 122.

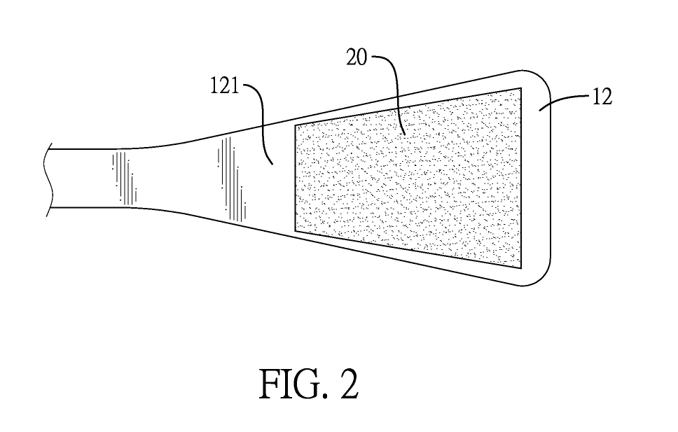

The piezochromic device 20 is mounted on the visible surface 121 of the work piece 12 of the instrument body 10. With reference to FIG. 2, the piezochromic device 20 can be a one-piece structure. When the piezochromic device 20 doesn't sense pressure, a first color is shown. When the piezochromic device 20 senses pressure, a pressed portion of the piezochromic device 20 shows a second color, which is different from the first color. When the pressure is removed from the piezochromic device 20, the piezochromic device 20 doesn't sense pressure and shows the first color. Therefore, the piezochromic device 20 senses the pressure to perform color change.

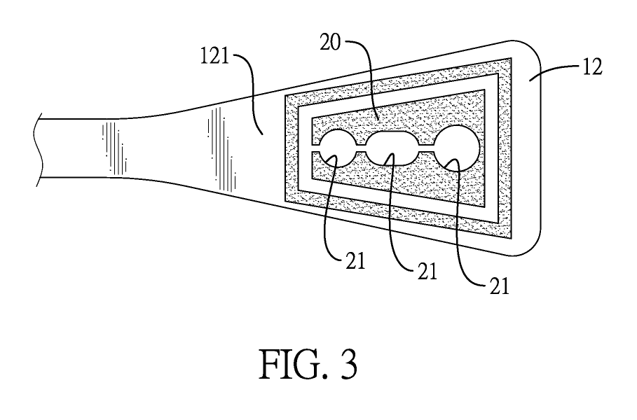

In addition, the piezochromic device 20 can be a hollow pattern structure, including one or more hollows. With reference to FIG. 3, the piezochromic device 20 includes multiple hollows 21. The hollows 21 are reference points to determine positions of the piezochromic device 20. As the embodiment shown in FIG. 3, the hollows 21 are aligned and respectively formed in different shapes (such as, for example, a circular shape, an oval shape, a polygonal shape, etc) enabling the doctor to distinguish a front section, a middle section or a rear section of the piezochromic device 20.

Since the piezochromic device 20 is mounted on the visible surface 121 of the work piece 12, the doctor can directly see the piezochromic device 20 during the surgery to determine whether the color change of the piezochromic device 20 occurs. Therefore, when the piezochromic device 20 contacts other surgical instruments or presses the normal organs or the normal tissues within the patient's body, the piezochromic device 20 senses the pressure and directly changes its color. When the doctor observes that the color of the piezochromic device 20 is changed, the doctor can adjust the movement of the surgical instrument and the surgery can be performed smoothly.

What is claimed is:

1. A surgical instrument with pressure sensing and vision feedback functions, comprising:
an instrument body including a work piece with a visible surface; and
a piezochromic device disposed on the visible surface of the work piece of the instrument body, wherein the piezochromic device performs a color change when the piezochromic device senses pressure.

2. The surgical instrument with pressure sensing and vision feedback functions of claim 1, wherein the piezochromic device is structured as one piece.

3. The surgical instrument with pressure sensing and vision feedback functions of claim 1, wherein the piezochromic device includes hollowed-out patterns.

4. The surgical instrument with pressure sensing and vision feedback functions of claim 3, wherein the piezochromic device includes a plurality of hollows.

5. The surgical instrument with pressure sensing and vision feedback functions of claim 4, wherein the plurality of hollows are different shapes.

6. The surgical instrument with pressure sensing and vision feedback functions of claim 4, wherein the plurality of hollows are aligned.

7. The surgical instrument with pressure sensing and vision feedback functions of claim 5, wherein the plurality of hollows are aligned.
